(19) United States
(12) Patent Application Publication
(10) Pub. No.: US 2013/0158439 A1
(43) Pub. Date: Jun. 20, 2013

(54) UPPER LIMB IMPAIRMENT MEASUREMENT SYSTEM

(71) Applicant: Steven Donald CLARKE, Shenton Park (AU)

(72) Inventor: Steven Donald CLARKE, Shenton Park (AU)

(21) Appl. No.: 13/719,722

(22) Filed: Dec. 19, 2012

Related U.S. Application Data
(60) Provisional application No. 61/600,756, filed on Feb. 20, 2012.

(30) Foreign Application Priority Data
Dec. 19, 2011 (AU) 2011905303

(51) Int. Cl. A61B 5/11

(52) U.S. Cl. CPC A61B 5/11
USPC 600/595

(57) ABSTRACT

A method of permanent impairment assessment of a person's upper limb and/or hand is implemented using a proprietary computer program, and comprises the step of displaying a digital image of the person's injured limb and/or hand 24 and of their uninjured limb and/or hand 22 on a visual display screen. A measurement tool 20 is provided on-screen for measuring the extent of injury, wherein the measurement tool can be moved over the digital image of both hands. The extent of injury of the injured limb and/or hand is measured by moving the measurement tool 20 so as to overlay the injured limb and/or hand 24. A value for the degree of impairment can be obtained using this measurement.

### FIELD OF THE INVENTION

The present invention relates to a method and apparatus for measuring upper limb permanent impairment and relates particularly, though not exclusively, to such a method and apparatus for measuring hand impairment due to digit amputation.

### BACKGROUND TO THE INVENTION

The standard text employed in Australia and in the USA for amputation assessment, and indeed for Permanent Impairment assessment overall, is the American Medical Association (AMA) “Guides to the Evaluation of Permanent Impairment” by Robert D. Rondinelli, which is currently in its 6^{th} Edition. In Western Australia the 5^{th} Edition (hereinafter “AMA 5^{th}”) is mandated by Workcover WA as the applicable standard for Permanent Impairment assessment. In other States of Australia the 4^{th} Edition (hereinafter “AMA 4^{th}”) is used. It specifies the way in which such measurements must be done with various formulae, tables and figures which rely on measurement in some cases and in ‘Diagnostic related Entities’ (used, for example, in Spine and lower limb assessments) amongst others.

In the prior art method of digit amputation assessment use is made of FIGS. 16.4 and 16.5 (or 16.6 and 16.7) in AMA 5^{th}. In AMA 4^{th} the corresponding figures in Chapter 3 are FIG. 7 (thumb) and FIG. 17 (fingers). A problem with using these figures from the AMA 5^{th} or 4^{th} is that they rely on clinical judgement, and the results are not reproducible, verifiable or transparent to any 3^{rd} party who may have an interest in the outcome of such an impairment assessment. In the Workers Compensation area, MVIT and similar arenas, the decision about an individual's degree of permanent impairment has significant implications for monetary compensation or in other cases an individual's access to a common law remedy. The existing method of assessment relies on a clinician's judgement as to the level of amputation compared against FIGS. 16.4 and 16.5 (or 16.6 and 16.7) in AMA 5^{th}, and is therefore somewhat subjective. There is a substantial risk that the clinician's judgement may be inaccurate or influenced by extraneous factors when making an assessment of the degree of impairment.

The present invention was developed with a view to providing a method and apparatus for measuring upper limb permanent impairment that is more precise and more accurate.

References to prior art in this specification are provided for illustrative purposes only and are not to be taken as an admission that such prior art is part of the common general knowledge in Australia or elsewhere.

### SUMMARY OF THE INVENTION

According to one aspect of the present invention there is provided a method of permanent impairment assessment of a person's upper limb and/or hand, the method comprising the steps of:

displaying a digital image of the person's injured limb and/or hand and of their uninjured limb and/or hand on a visual display screen;

providing a measurement tool on-screen for measuring the extent of injury, wherein the measurement tool can be moved over the digital image;

a user measuring the extent of injury of the injured limb and/or hand by moving the measurement tool so as overlay the injured limb and/or hand; and,

obtaining a value for the degree of impairment using this measurement.

Preferably the method further comprises the steps of:

the user identifying in the digital image key scaling markers on the uninjured limb and/or hand and any corresponding key scaling markers remaining on the injured limb and/or hand; and,

scaling the measurement tool using the key scaling markers on the uninjured limb and/or hand.

Advantageous the method further comprises the step of the user physically marking the key scaling markers on both the uninjured hand and the injured hand, prior to the step of making the digital image to aid in the positive identification of such key scaling markers in the digital image.

Preferably the method further comprises the step of saving a digital record of the digital image with measurements superimposed on the image.

Preferably, once the measurement tool has been scaled, the digital image of the uninjured limb and/or hand is marked with a visual scale indicator representative of the scale resolution. Preferably the visual scale indicator is superimposed on the digital image of the uninjured limb and/or hand, and a permanent copy of this image is stored with the patient record to provide a visual indicator that the scale of the measurement tool remained unchanged between measurements. Preferably an image of the measurement tool overlaying the injured limb and/or hand is also superimposed on the permanent copy of the digital image of the injured hand stored with the patient record. Preferably this image also includes the digital scale indicator, providing visual proof that the scale of the measurement tool was unchanged between measurements.

Preferably the method also comprises the step of enabling the user to select the types of measurement of permanent impairment it is desired to make and on which parts of the upper limbs and/or hands the measurements are to be made. Advantageously the method also comprises the step of indicating the number and types of views (digital images) required of the upper limbs and/or hands to perform the selected measurements.

Typically the step of obtaining a value for the degree of permanent impairment using the tool may be done visually on-screen, by reading the percentage directly off the visual representation of the tool on-screen. Alternatively, or in addition, the tool can also automatically calculate the degree of permanent impairment. Advantageously, with the tool correctly scaled and aligned, the user merely clicks on the actual point of impairment on the digital image and the tool can calculate the percentage impairment using a stored look-up table.

According to another aspect of the present invention there is provided an apparatus for permanent impairment assessment of a person's upper limb and/or hand, the apparatus comprising:

a visual display screen for displaying a digital image of the person's injured limb and/or hand and of their uninjured limb and/or hand;

digital processing means for processing the digital image and controlling the information displayed in the visual display screen;

an on-screen measurement tool for measuring the extent of the injury, wherein the measurement tool can be moved over the digital image; and,

wherein, in use, the extent of injury of the injured limb and/or hand can be measured by moving the measurement tool so as overlay the injured limb and/or hand and this measurement can be used to obtain a value for the degree of impairment.

Preferably the apparatus further comprises means for identifying in the digital image key scaling markers on the uninjured limb and/or hand and any corresponding key scaling markers remaining on the injured limb and/or hand, and wherein the measurement tool can be scaled using the key scaling markers on the uninjured limb and/or hand.

Advantageously the apparatus further comprises imaging means for making a digital image of the person's injured limb and/or hand and of their uninjured limb and/or hand.

According to a still further aspect of the present invention there is provided a method of permanent impairment assessment of a person's rotated digit or thumb, the method comprising the steps of:

having the person grip an elongate linear object with both hands;

measuring a first angle of rotation, if any, of the person's uninjured digit or thumb;

measuring a second angle of rotation of the person's injured digit or thumb; and,

subtracting the first angle from the second angle to obtain a measurement of the degree of rotation of the injured digit or thumb; and,

obtaining a value for the degree of impairment using this measurement.

Preferably the angles of rotation are measured by drawing an imaginary line between the nail folds on the respective digits or thumbs. Preferably the angles of rotation are measured using an edge of the elongate linear object as a reference base line.

Advantageously the method further comprises the steps of:

displaying a digital image of the person's injured hand and of their uninjured hand gripping the elongate object on a visual display screen;

providing a measurement tool on-screen for measuring the angle of rotation, wherein the measurement tool can be moved over the digital image;

measuring the first and second angles of rotation of the uninjured digit or thumb and injured digit or thumb respectively by moving the measurement tool so as to first overlay the uninjured digit or thumb and then the injured digit or thumb.

Preferably the measurement tool is in the form of an on-screen protractor which can be used to measure the angle of rotation of the injured thumb or digit shown in the image.

According to yet another aspect of the present invention there is provided an apparatus for permanent impairment assessment of a person's rotated digit or thumb, the apparatus comprising:

an elongate linear object adapted to be gripped with both hands; and,

means for measuring a first angle of rotation, if any, of the person's uninjured digit or thumb, and for measuring a second angle of rotation of the person's injured digit or thumb;

wherein, in use, by subtracting the first angle from the second angle to obtain a measurement of the degree of rotation of the injured digit or thumb, the degree of impairment can also be obtained using this measurement.

Preferably the elongate linear object is a cylindrical object that can be comfortably gripped with both hands. Typically the cylindrical object has an external diameter of between 40-50 mm.

Throughout the specification, unless the context requires otherwise, the word “comprise” or variations such as “comprises” or “comprising”, will be understood to imply the inclusion of a stated integer or group of integers but not the exclusion of any other integer or group of integers. Likewise the word “preferably” or variations such as “preferred”, will be understood to imply that a stated integer or group of integers is desirable but not essential to the working of the invention.

### BRIEF DESCRIPTION OF THE DRAWINGS

The nature of the invention will be better understood from the following detailed description of several specific embodiments of the method and apparatus for measuring upper limb permanent impairment, given by way of example only, with reference to the accompanying drawings, in which:

FIG. 1 is a flowchart illustrating a preferred embodiment of the computer software program for implementing a method of measuring upper limb permanent impairment according to the present invention;

FIG. 2 is a block diagram illustrating a preferred embodiment of an apparatus for measuring upper limb permanent impairment according to the present invention;

FIG. 3 illustrates a typical on-screen dialogue box employed in the computer software program illustrated in FIG. 1;

FIG. 4a illustrates a typical screen shot employed in the computer software program illustrated in FIG. 1, showing a digital image of a pair of hands with digit amputation on one hand;

FIG. 4b illustrates a typical screen shot employed in the computer software program illustrated in FIG. 1, showing an x-ray image of a pair of digits with partial amputation of one digit;

FIG. 5a illustrates a typical screen shot employed in the computer software program illustrated in FIG. 1, showing a digital image of a pair of thumbs with partial amputation of one thumb;

FIG. 5b illustrates a typical screen shot employed in the computer software program illustrated in FIG. 1, showing a digital x-ray image of a pair of thumbs with partial amputation of one thumb;

FIG. 6 illustrates a typical screen shot optionally employed in the computer software program illustrated in FIG. 1, showing a digital image of a hand for measuring the degree of rotation of a digit;

FIG. 7 illustrates a typical screen shot optionally employed in the computer software program illustrated in FIG. 1, showing a digital image of a hand for measuring the degree of rotation of a thumb;

FIG. 8 illustrates a typical screen shot optionally employed in the computer software program illustrated in FIG. 1, showing a digital image of both an injured and uninjured hand for measuring the degree of digit angulation;

FIG. 9 illustrates a typical screen shot optionally employed in the computer software program illustrated in FIG. 1, showing a series of digital images of a thumb and hand for measuring degree of thumb motion;

FIGS. 10 and 11 illustrate typical screen shots optionally employed in the computer software program illustrated in FIG. 1, showing a digital image of both an injured and uninjured hand for measuring the degree of thumb adduction and opposition respectively;

FIG. 12 illustrates a typical screen shot optionally employed in the computer software program illustrated in FIG. 1, showing a series of digital images of a hand for measuring finger motion;

FIG. 13 illustrates a typical screen shot optionally employed in the computer software program illustrated in FIG. 1, showing a series of digital images of a hand and wrist for measuring wrist motion;

FIG. 14 illustrates a typical screen shot optionally employed in the computer software program illustrated in FIG. 1, showing a series of digital images of an upper limb and torso for measuring elbow motion; and,

FIG. 15 illustrates a typical screen shot optionally employed in the computer software program illustrated in FIG. 1, showing a series of digital images of an upper limb and torso for measuring shoulder motion.

### DETAILED DESCRIPTION OF PREFERRED EMBODIMENTS

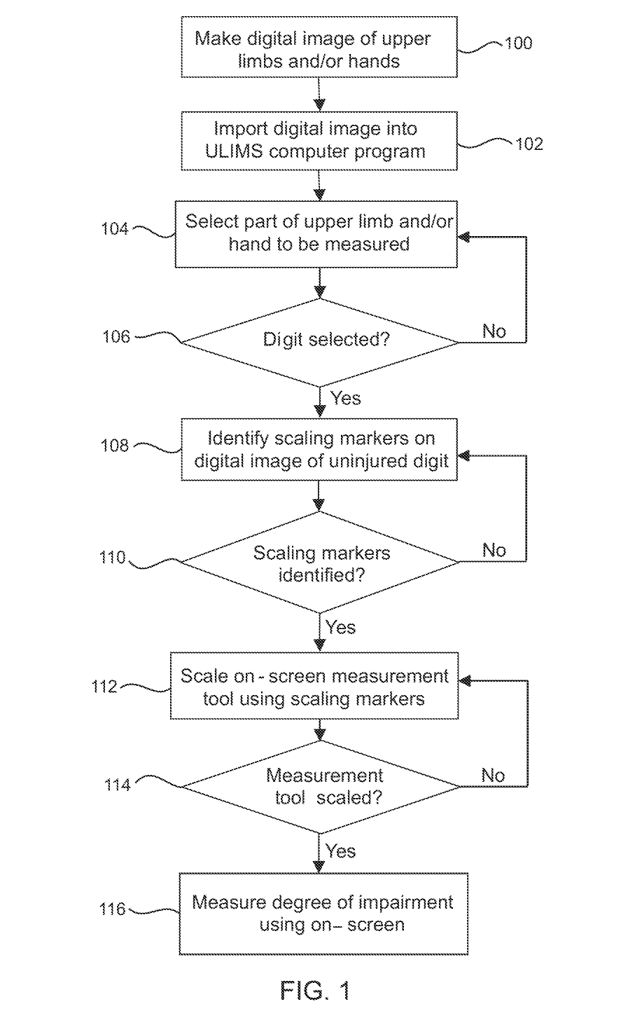

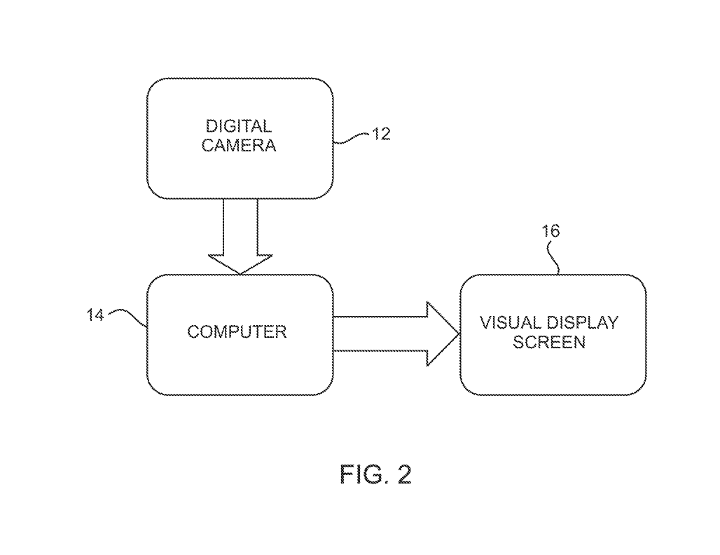

A preferred embodiment of the method of measuring upper limb permanent impairment in accordance with the invention, as illustrated in FIGS. 1 to 12, is implemented using a proprietary computer program for Upper Limb Impairment Measurement System (U.L.I.M.S.), and typically comprises the steps of making a digital image of the person's injured limb and/or hand and of their uninjured limb and/or hand (step 100 in FIG. 1). Typically the digital image is made using a bench-mounted digital camera 12 which can be connected directly to a computer 14, as shown in FIG. 2. It is also possible for the digital image to be imported from another source. For example, someone may take a digital photograph at another time and place, and subsequently forward it to the computer 14 via the Internet. The computer 14 may be any suitable computing device with digital processing capacity capable of running the ULIMS software, such as a laptop or desktop computer, a digital mobile phone or tablet device. A visual display screen 16 is connected to the computer 14 for displaying the digital image.

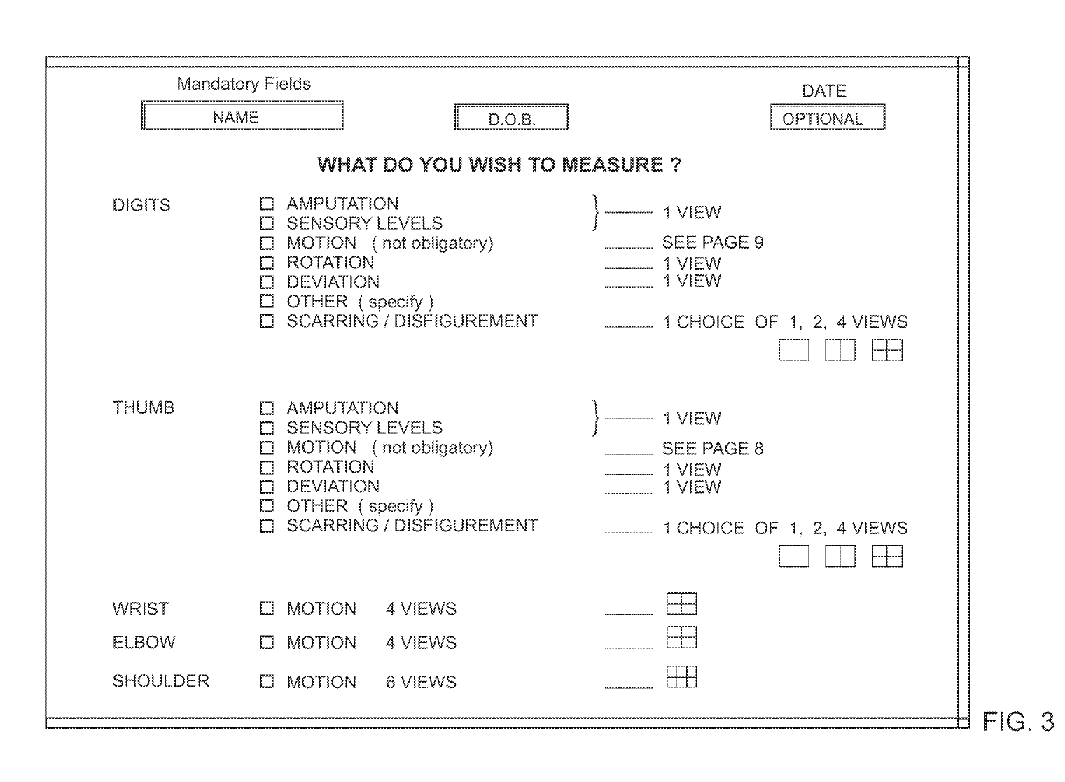

Typically the ULIMS software imports the digital image ready for display on the visual display screen 16, (step 102 in FIG. 1). Typically the ULIMS software provides a dialogue box to allow the operator to select which part of an upper limb and/or hand it is desired to be measured (step 104 in FIG. 1). FIG. 3 illustrates a typical dialogue box of this nature, which allows the operator to specify the types of measurement of permanent impairment it is desired to make and on which parts of the upper limbs and/or hands (digit, thumb, wrist, elbow, shoulder, etc) the measurements are to be made. This dialogue box also indicates the number and types of views (digital images) required of the upper limbs and/or hands to perform the selected measurements.

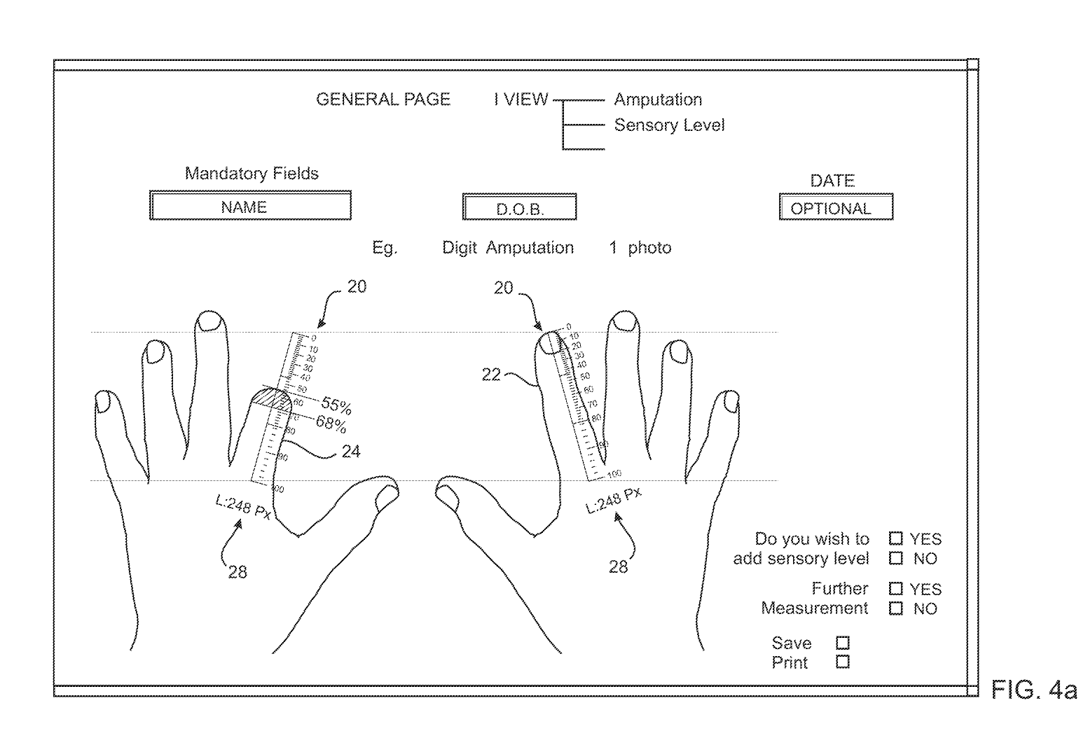

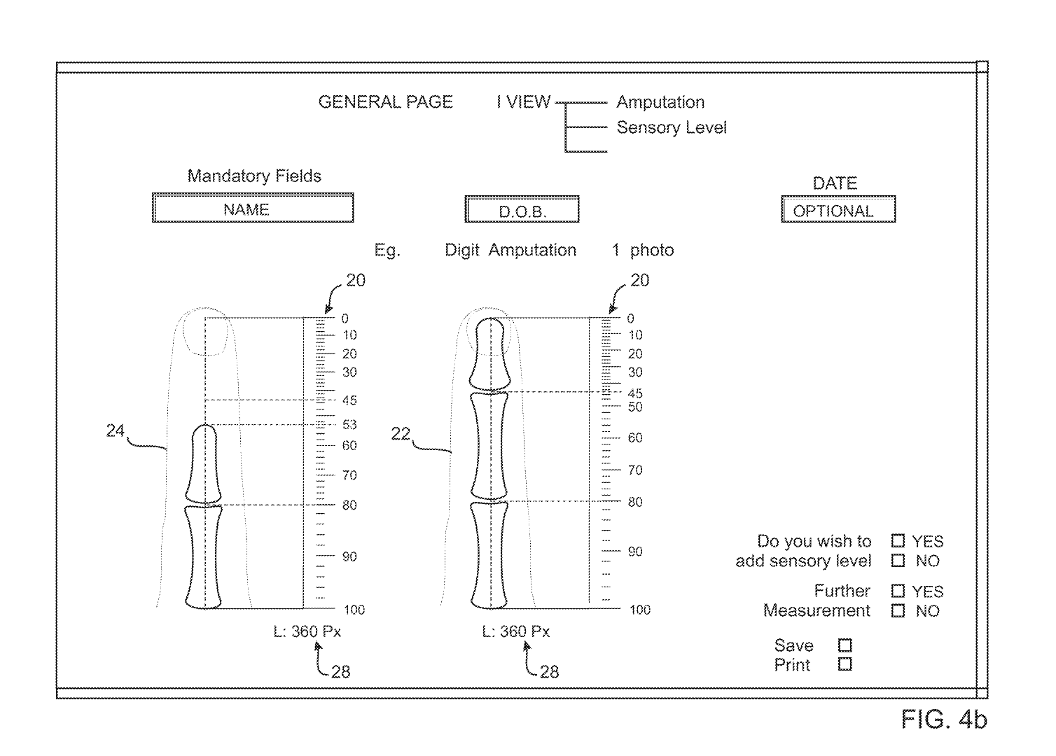

Assuming the operator selects digit amputation, (step 106 in FIG. 1) the software then displays the digital image of both the injured and uninjured hand on the visual display screen 16, as shown in FIG. 4a. The operator is then typically required to visually identify in the digital image key scaling markers on the uninjured hand (or digit) and any corresponding key scaling markers remaining on the injured hand (or digit) (step 108 in FIG. 1). To aid in the positive identification of such key scaling markers in the digital image, it may be advantageous to physically mark the relevant points with a black marker on both the uninjured hand and the injured hand, prior to the step of making the digital image. In the illustrated example, the index finger on the left hand has been partially amputated. According to the principles of amputation impairment, described in AMA 5^{th} (and AMA 4^{th}), amputation through each joint level of a digit is given a relative value of loss to the entire digit as follows: digit metacarpophalangeal (MP) joint, 100%; thumb interphalangeal (IP) joint, 50%; finger proximal interphalangeal (PIP) joint, 80%; and finger distal interphalangeal (DIP) joint, 45%.

The present inventor has observed that in the case of digital joint fusion or fracture, there is often loss of length after healing, equivalent to amputation. The degree of impairment for such ‘amputation equivalent’ cases can also be assessed using the same procedure as for digital amputation in the preferred embodiment of the ULIMS software

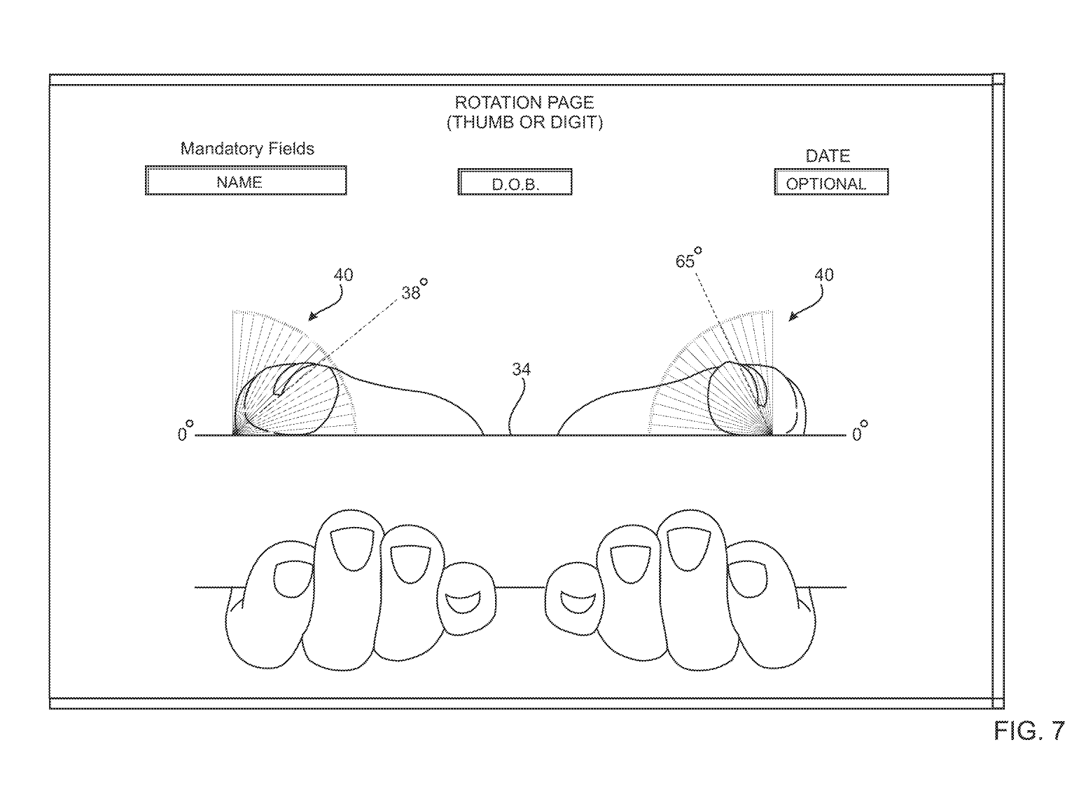

The finger MP (100%), PIP (80%) and DIP (45%) joints, together with the tip of the finger (0%) are typically identified as key scaling markers on the digital image of the uninjured right hand (step 110 in FIG. 1). The ULIMS software is programmed to provide a measurement tool 20 on-screen, (see FIG. 4a) for measuring the extent of the injury. The measurement tool 20 can be moved on-screen, for example by using a mouse, so as to overlay the digital image of the uninjured index finger 22 of the right hand. The measurement tool in this embodiment is in the form of a scalable and rotatable rule 20 and provides a measurement of the percentage of digit impairment. The scalable rule 20 is derived from the underlying principles of digit impairment as illustrated in the graph shown in FIG. 16-7 of AMA 5^{th}.

The measurement tool 20 can then be scaled using the key scaling markers on the uninjured index finger 22, (step 112 in FIG. 1) by aligning the tip and the MP joint of the finger 22 with the “0%” and the “100%” impairment levels respectively on the measurement tool 20, as shown on the right hand in FIG. 4a. Preferably, once the measurement tool 20 has been scaled, the digital image of the hand with the uninjured index finger 22 is marked with a visual scale indicator representative of the scale resolution. In the illustrated example the visual scale indicator is marked as “L:248 Px.” This digital scale indicator 28 is superimposed on the digital image of the injured hand, and a permanent copy of this image is stored with the patient record. This provides a visual indicator that the scale of the measurement tool 20 remains unchanged between measurements.

When the measurement tool 20 has thus been scaled, (step 114 in FIG. 1) it is possible to measure the degree of impairment of the index finger 24 on the injured left hand by moving the measurement tool 20 and rotating it so as to align with the key scaling markers on the left index finger 24 (step 116 in FIG. 1). Preferably an image of the measurement tool 20 overlaying the injured finger 24 is also superimposed on the permanent copy of the digital image of the injured hand stored with the patient record. As can be seen in FIG. 4a, this image also includes the digital scale indicator 28, providing visual proof that the scale of the measurement tool 20 was unchanged between measurements. In the illustrated example, the finger 24 has been amputated between the PIP and the DIP joints. However the measurement tool 20 can still be aligned with the MP and PIP joints, and in this case the degree of permanent impairment can then be calculated using the tool to be 55%.

Calculation of the degree of permanent impairment using the tool 20 may be done visually on-screen, by reading the percentage directly off the visual representation of the tool on-screen. Alternatively, or in addition, the tool 20 can also automatically calculate the degree of permanent impairment. With the tool correctly scaled and aligned, the operator merely clicks on the actual point of amputation on the digital image and the tool can calculate the percentage impairment using a look-up table stored in the software.

The same screen, showing the digital image of both hands as in FIG. 4a, may also be used to make several other measurements. For example, the tool 20 can also be used to calculate the level of sensory loss (either transverse or longitudinal), as well as the sensory level, with reference to FIG. 16.6 or 16.7 in AMA 5th or the equivalent in AMA 4th. The digital image of the two hands, with the scaled measurement tool 20 superimposed on the injured digit, together with the digital scale indicator and other measured values, can be saved and/or printed to provide a permanent record of the measurements. This permanent record can be presented as evidence in support of any subsequent claim for compensation or in a dispute regarding the degree of permanent impairment.

The same method and computer system can be used if x-ray images are available of the amputated digit to measure the degree of permanent impairment. The software displays the digital x-ray image of both the injured and uninjured digit on the visual display screen 16, as shown in FIG. 4b. The operator is then typically required to visually identify in the digital image key scaling markers on the uninjured digit and any corresponding key scaling markers remaining on the injured digit, as before. In this case the finger MP (100%), PIP (80%) and DIP (45%) joints, together with the tip of the finger (0%) can be more accurately identified as key scaling markers on the digital image of the uninjured right digit.

When the measurement tool 20 has been scaled, (step 114 in FIG. 1) it is possible to measure the extent of the injury of the index finger 24 on the injured left hand by moving the measurement tool so as to align with the key scaling markers on the left digit 24 (step 116 in FIG. 1). In the illustrated example, (see FIG. 4b) the finger 24 has been amputated between the PIP and the DIP joints. However the measurement tool 20 can still be aligned with the MP and PIP joints, and in this case the degree of permanent impairment can then be more accurately calculated using the tool to be 53%.

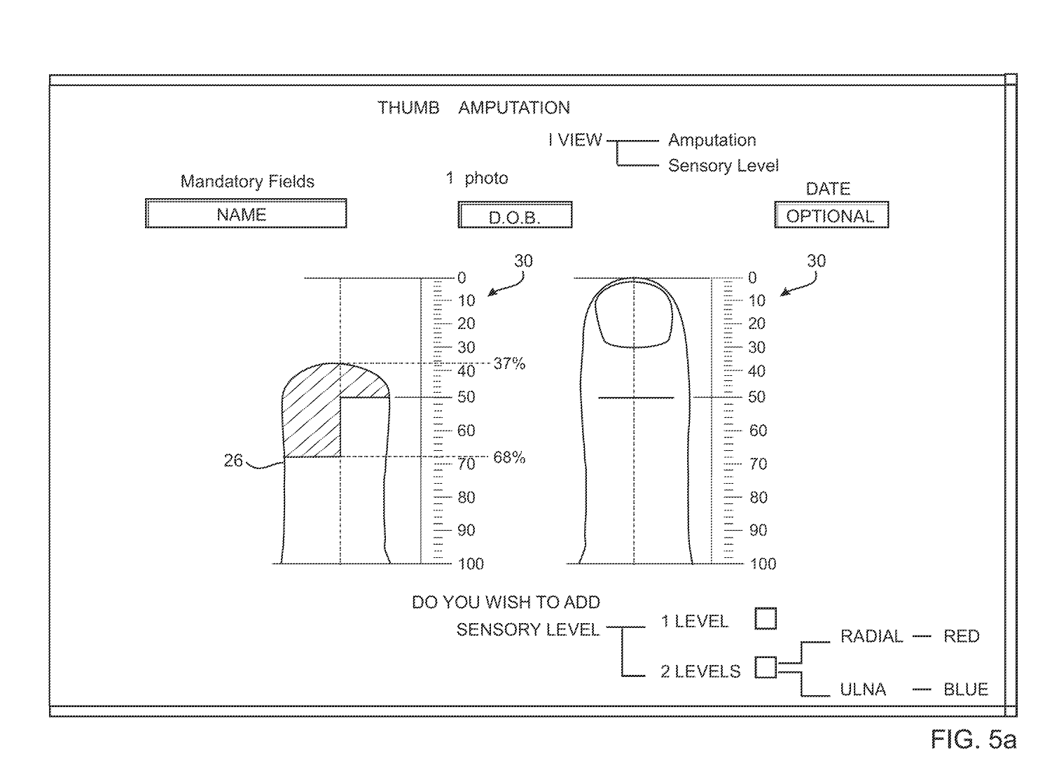

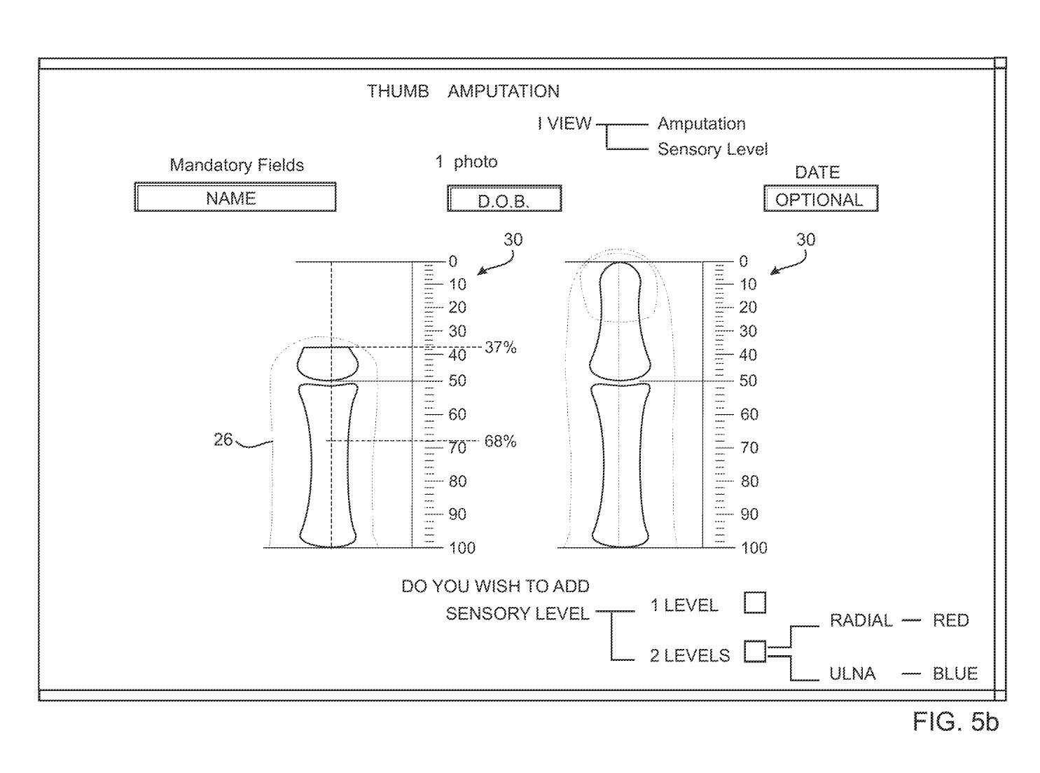

The method of measuring the degree of permanent impairment of an amputated thumb is very similar to that described above in relation to an amputated finger. FIG. 5a illustrates a typical digital image of the left and right thumbs of a person who whose left thumb 26 has been amputated between the IP joint and the tip of the thumb 26. The same sequence of steps, as illustrated in FIG. 1, is implemented using the ULIMS software. The measurement tool in this case is again in the form of a scalable rule 30, which is derived from the underlying principles of thumb impairment as illustrated in the graph shown in FIG. 16.4 or 16.6 in AMA 5^{th} or FIG. 7 in AMA 4^{th}. The software may use a different look-up table to automatically calculate the degree of permanent impairment.

As with digital amputation, the same method can also be used if x-ray images are available of the amputated thumb to measure the degree of permanent impairment. The software displays the digital x-ray image of both the injured and uninjured thumb on the visual display screen 16, as shown in FIG. 5b. When measuring thumbs, only one key scaling marker (IP joint 50%) needs to be used for scaling the rule 30. The tip of the thumb is assigned 0% and the MP joint is assigned 100%.

Longitudinal sensory loss measurements, based on the sensory function of the radial and ulnar side of the digit, can also be made, using the two-point discrimination test, and recorded in conjunction with the digital image to provide a permanent record of the measurements.

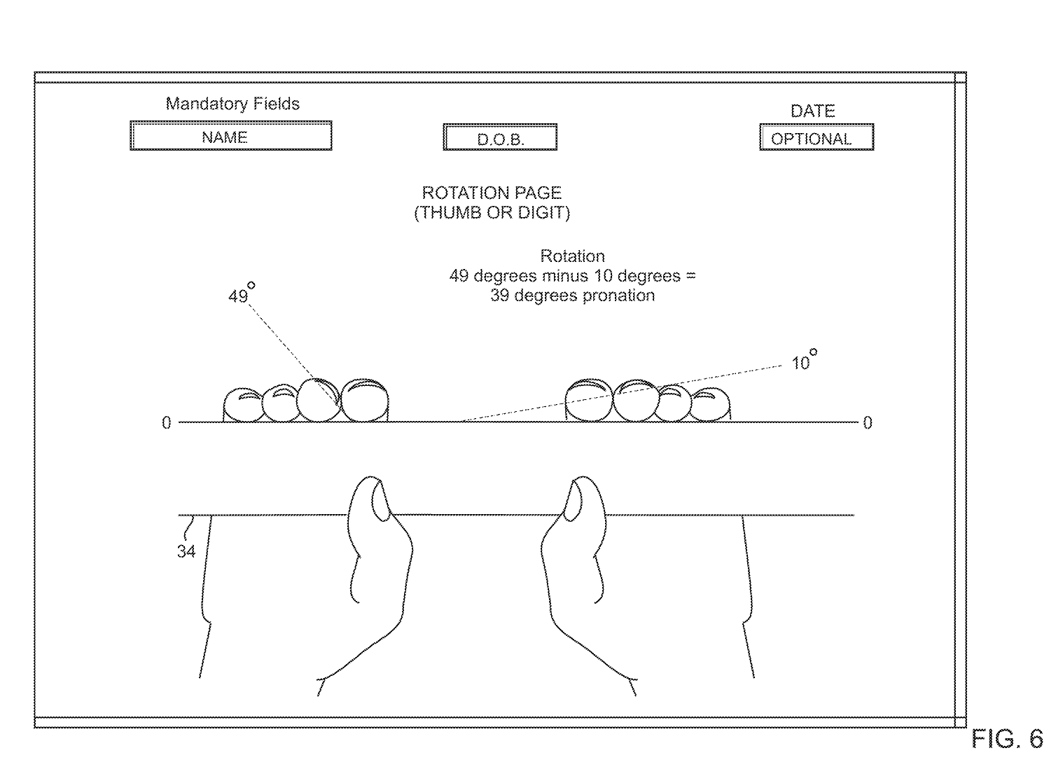

The ULIMS software can also optionally incorporate various additional measurements of upper limb permanent impairment. FIGS. 6 to 15 will be used to describe some of these additional measurements.

FIGS. 6 and 7 illustrate on-screen digital images of an injured hand and an uninjured hand used to measure the degree of rotation of an injured digit or thumb. In order to make a suitable digital image of the digit or thumb, the person is preferably required to hold an elongate linear object with both hands as shown in FIGS. 6 and 7. Preferably the elongate linear object is in the form of a grip tube 34, selected to have a diameter that enables the alignment of the respective digits to be clearly visible in the digital image. The tube also ensures the digit joints are in a degree of flexion suitable for this kind of measurement. Preferably the grip tube 34 has an external diameter of about 40-50 mm. Here again the ULIMS software provides an on-screen measurement tool 40 (see FIG. 7) for measuring the degree of rotation.

The measurement tool in this case is in the form of an on-screen protractor 40 which can be used to measure the angle of rotation of the injured thumb or digit shown in the image. As with the amputation assessment, the measurement tool can be moved over the digital image, and, the degree of rotation of the injured thumb or digit can be measured by locating the measurement tool on-screen so as overlay the injured thumb or digit.

So, for example, when measuring the degree of rotation of the injured digit on the left hand shown in FIG. 6, the measurement tool is first moved to overlay the corresponding uninjured digit on the right hand. The angle of rotation is measured by drawing an imaginary line between the nail folds on the uninjured digit, and using an edge of the grip tube 34 as a reference base line. This indicates a first angle of rotation of 10°. The measurement tool is then moved to overlay the injured digit, and the angle of rotation is measured by drawing an imaginary line between the nail folds on the injured digit. This indicates a second angle of rotation of 49°.

A measurement of the degree of rotation of the injured digit can then be obtained by subtracting the first angle from the second angle to yield an angle of 39° pronation (49°-10°). This measurement can then be used to obtain the degree of impairment with reference to Table 16.21 in AMA 5^{th} or Table 3.22 in AMA 4^{th}. Alternatively, the ULIMS software may use another look-up table to automatically calculate the degree of permanent impairment. A similar process can be employed to calculate the degree of rotation of an injured thumb as shown in FIG. 7. In the illustrated embodiment, this yields a degree of thumb rotation of 27° (65°-38°).

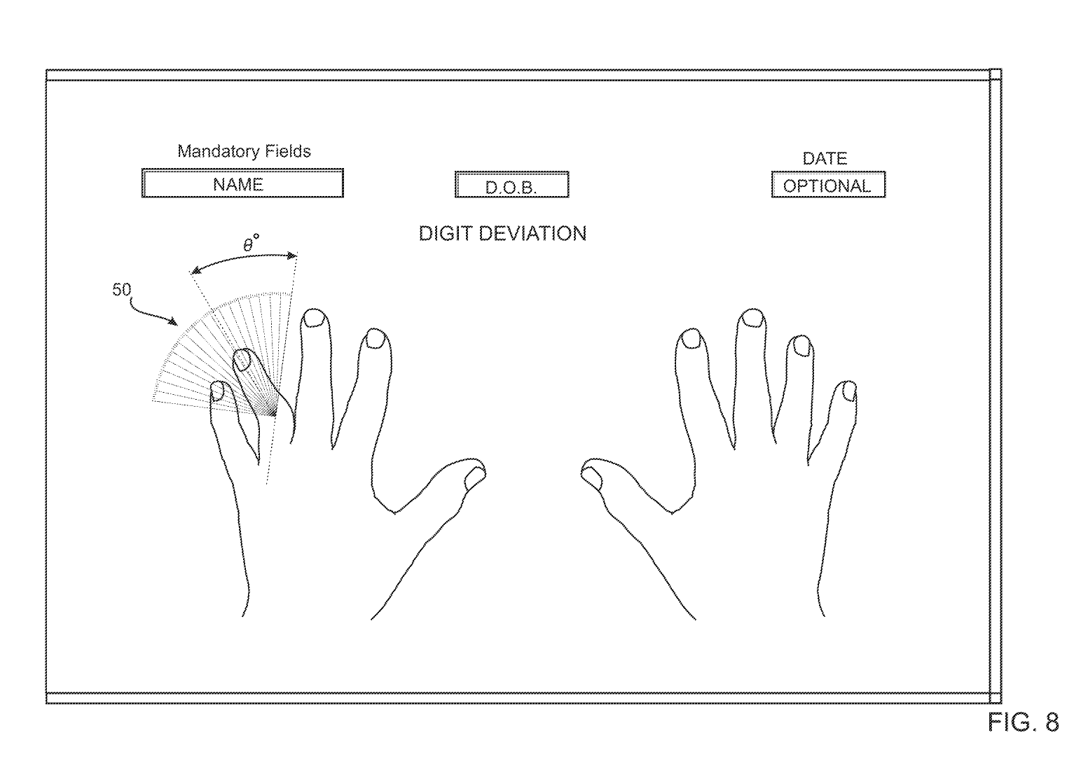

FIG. 8 illustrates a digital image provided on-screen for measuring and recording the degree of permanent impairment as a result of digit angulation (also referred to as “active ulnar or radial deviation”). Here again the ULIMS software provides an on-screen measurement tool 50 for measuring the degree of angulation. The measurement tool 50 in this case takes the form of an on-screen protractor 50 which can be used to measure the angle of deviation of the injured digit shown in the image, compared to the uninjured digit. As with the amputation assessment, the measurement tool can be moved over the digital image, and, the degree of angulation of the injured digit can be measured by locating the measurement tool on-screen so as overlay the injured digit. Based on this measurement, the degree of impairment can then be obtained with reference to Table 16.20 in AMA 5^{th} or Table 3.21 in AMA 4^{th}. Alternatively, the ULIMS software uses another look-up table to automatically calculate the degree of permanent impairment.

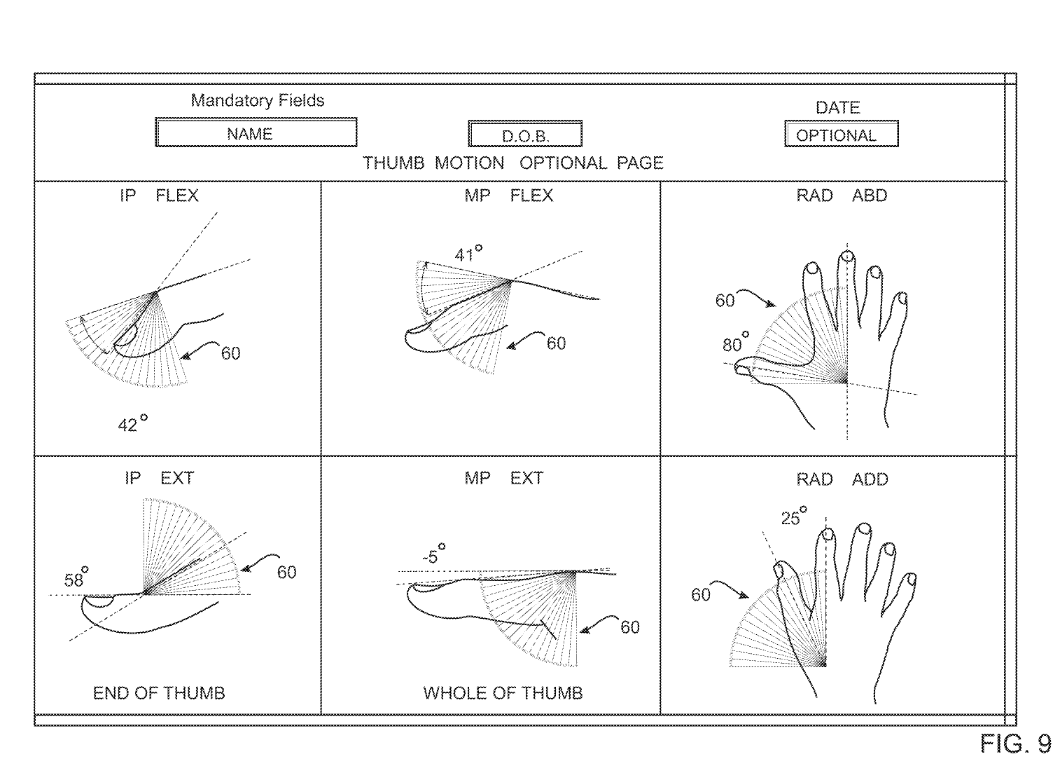

FIG. 9 illustrates a series of digital images provided on-screen for measuring and recording the degree of permanent impairment in thumb motion. Seven different views of the thumb are shown for measuring IP joint flexion, MP joint flexion, CMC joint radial abduction, opposition, IP joint extension, MP joint extension and CMC adduction. Here again the ULIMS software provides an on-screen measurement tool, typically in the form of a protractor 60, for measuring the extent of the injury. As with the amputation assessment, the measurement tool 60 can be moved over the digital image, and, the extent of injury of the injured limb and/or hand can be measured by locating the measurement tool on-screen so as overlay the injured hand or thumb. The degree of impairment can then be obtained using this measurement.

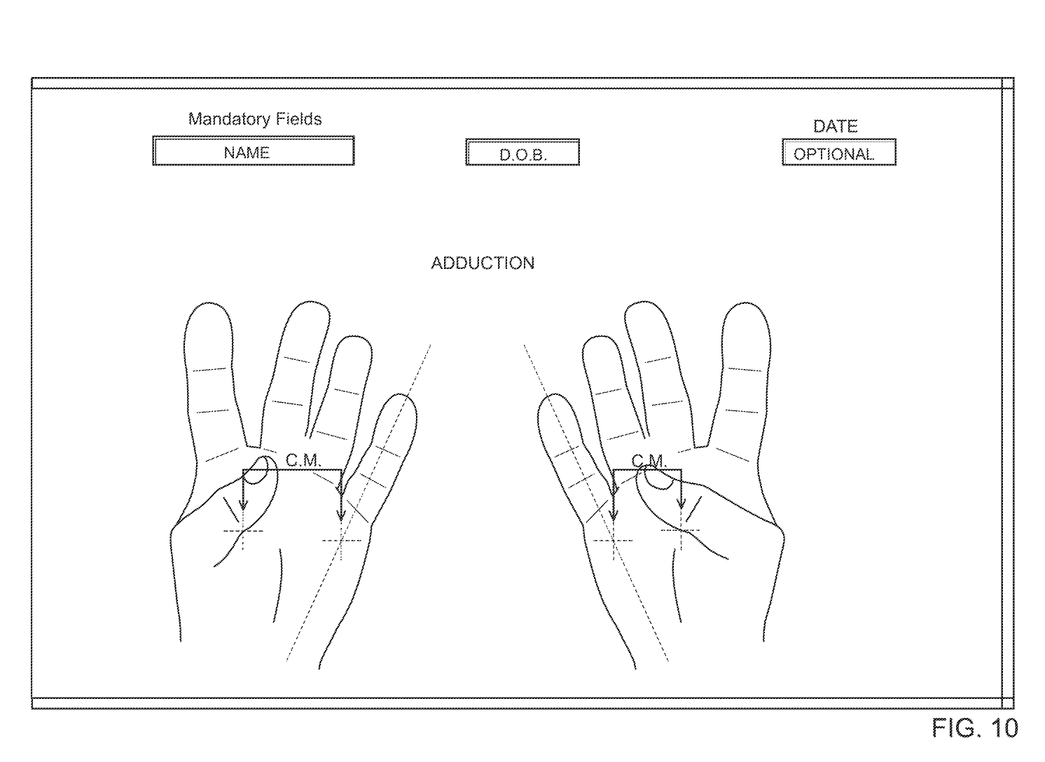

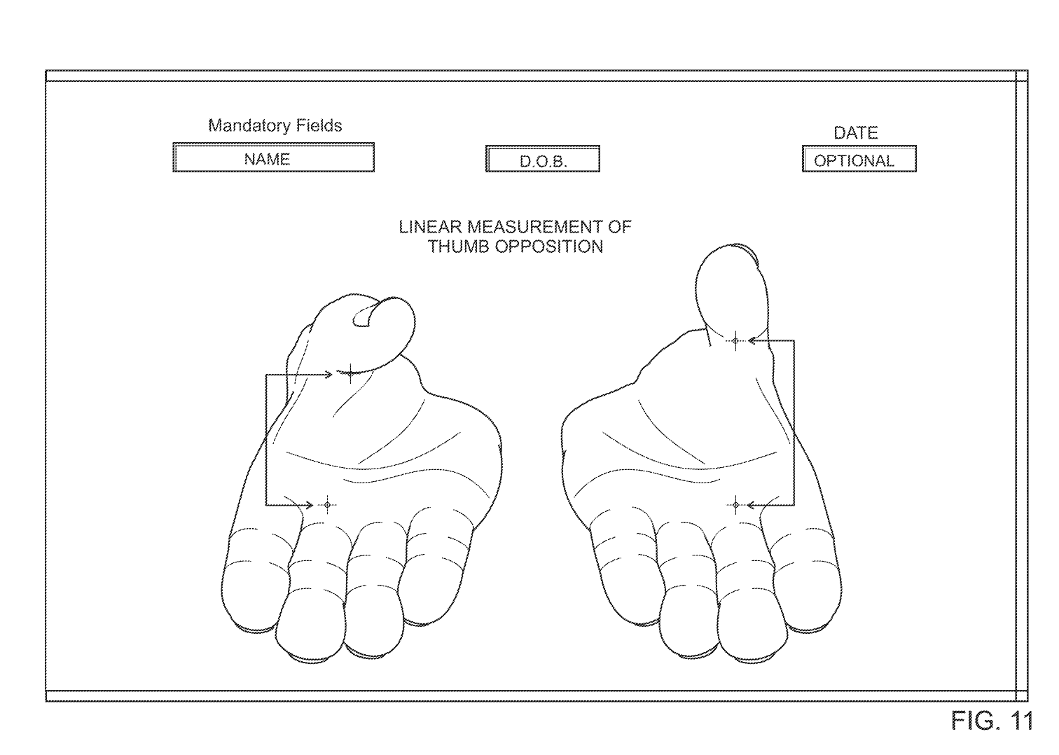

FIGS. 10 and 11 illustrate two different views of a pair of hands for measuring the degree of impairment of thumb adduction and opposition respectively. Here again the ULIMS software provides an on-screen measurement tool, in the form of a rule, for measuring the degree of impairment. As with the amputation assessment, the measurement tool can be moved over the digital image, and, the degree of impairment of the injured thumb can be measured by locating the measurement tool on-screen so as overlay the injured thumb and hand.

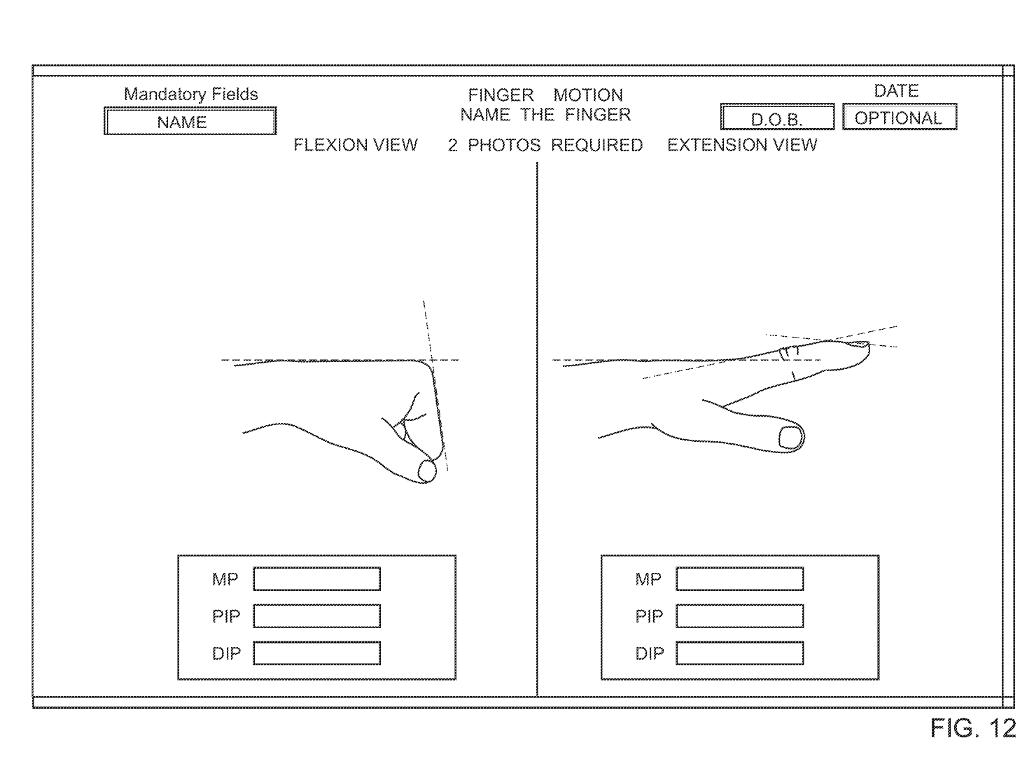

FIG. 12 illustrates a series of digital images provided on-screen for measuring and recording the degree of permanent impairment in finger (or thumb) motion. Two different views of the finger are shown for measuring finger flexion and extension respectively. The fingers have two functional units of motion associated with each joint; each joint is assigned the same relative functional value as that employed in amputation impairment assessment. Here again the ULIMS software provides an on-screen measurement tool, in the form of a goniometer, (not illustrated) for assessing the extent of injury by measuring the angles of motion, both in flexion and extension, at each joint.

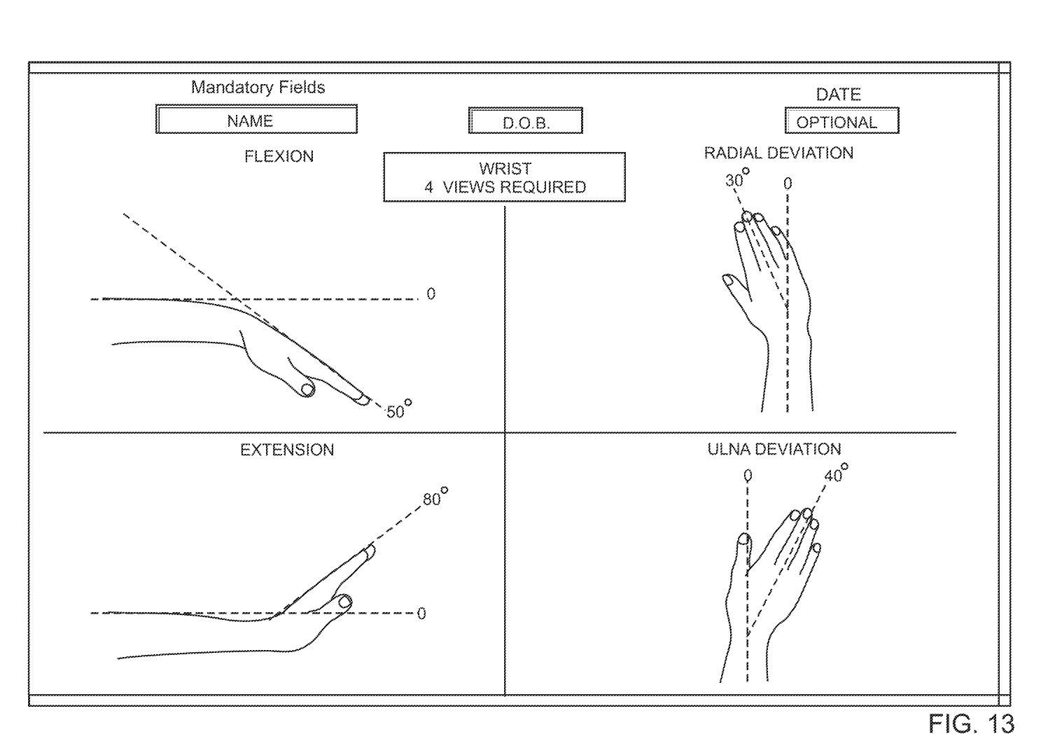

As with the amputation assessment, the measurement tool can be moved over the digital image, and, the extent of injury of the injured finger can be measured by manipulating the measurement tool on-screen so as overlay the injured finger. This measurement can then be used to obtain the degree of impairment with reference to FIGS. 16-21, 16-23 and 16-25 in AMA 5^{th}, (or FIGS. 19, 21 and 23 in AMA 4^{th}) for calculating the degree of permanent finger impairments due to ankylosis, loss of extension and loss of flexion at the DIP joint, PIP joint and MP joint respectively. These values, together with the measured angles can be recorded together with the digital image and a hardcopy printed if desired as a permanent record. For impairment of thumb motion, the measurement of the extent of injury can be used to calculate the degree of impairment with reference to FIG. 16.12, FIG. 16.15 and Tables 16.8a, Table 16.8b and Table 16.9 in AMA 5^{th} (and FIGS. 10 and 13, and Tables 5, 6 and 7 in AMA 4^{th}). Alternatively the ULIM software may store several look-up tables in the computer memory to automatically calculate the degree of permanent impairment.

FIG. 13 illustrates a series of digital images provided on-screen for measuring and recording the degree of permanent impairment in wrist motion. Four different views of the injured hand and wrist (left or right) are shown for measuring wrist joint flexion, wrist joint extension, ulnar deviation (UD) and radial deviation (RD). Here again the ULIMS software provides an on-screen measurement tool (not shown) for measuring the extent of the injury. As with the amputation assessment, the measurement tool can be moved over the digital image, and, the angle of motion of the injured limb and/or hand can be measured by locating the measurement tool on-screen so as overlay the injured hand or thumb.

This measurement can then be used to obtain the degree of impairment with reference to FIGS. 16-28 and 16-31 in AMA 5^{th}, (or FIGS. 26 and 29 in AMA 4^{th}), for calculating the degree of permanent wrist motion impairments due to ankylosis, loss of extension, loss of flexion, loss of radial deviation, and loss of ulnar deviation at the wrist joint. These values, together with the measured angles can be recorded together with the digital image and a hardcopy printed if desired as a permanent record.

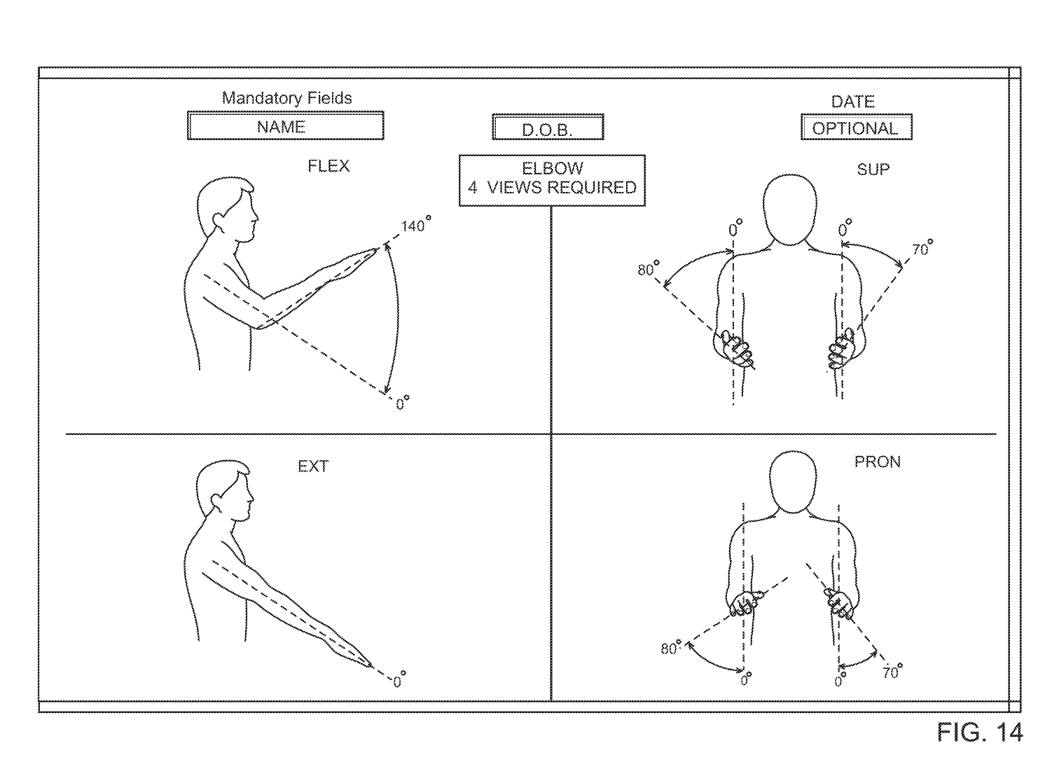

FIG. 14 illustrates a series of digital images provided on-screen for measuring and recording the degree of permanent impairment in elbow motion. Four different views of the injured elbow (left or right) are shown on-screen for measuring elbow joint flexion, elbow joint extension, supination and pronation. Here again the ULIMS software provides an on-screen measurement tool (not shown) for measuring the extent of the injury. As with the amputation assessment, the measurement tool can be moved over the digital image, and, the relevant angles of motion of the injured limb can be measured by locating the measurement tool on-screen so as overlay the injured elbow.

This measurement can then be used to obtain the degree of impairment with reference to FIGS. 16-34 and 16-37 in AMA 5^{th}, (or FIGS. 32 and 35 in AMA 4^{th}), for calculating the degree of permanent elbow motion impairments due to loss of extension, loss of flexion, loss of supination, loss of pronation, and ankylosis of the elbow joint. Alternatively the ULIM software may store several look-up tables in the computer memory to automatically calculate the degree of permanent impairment. These values, together with the measured angles can be recorded in conjunction with the digital image and a hardcopy printed if desired as a permanent record.

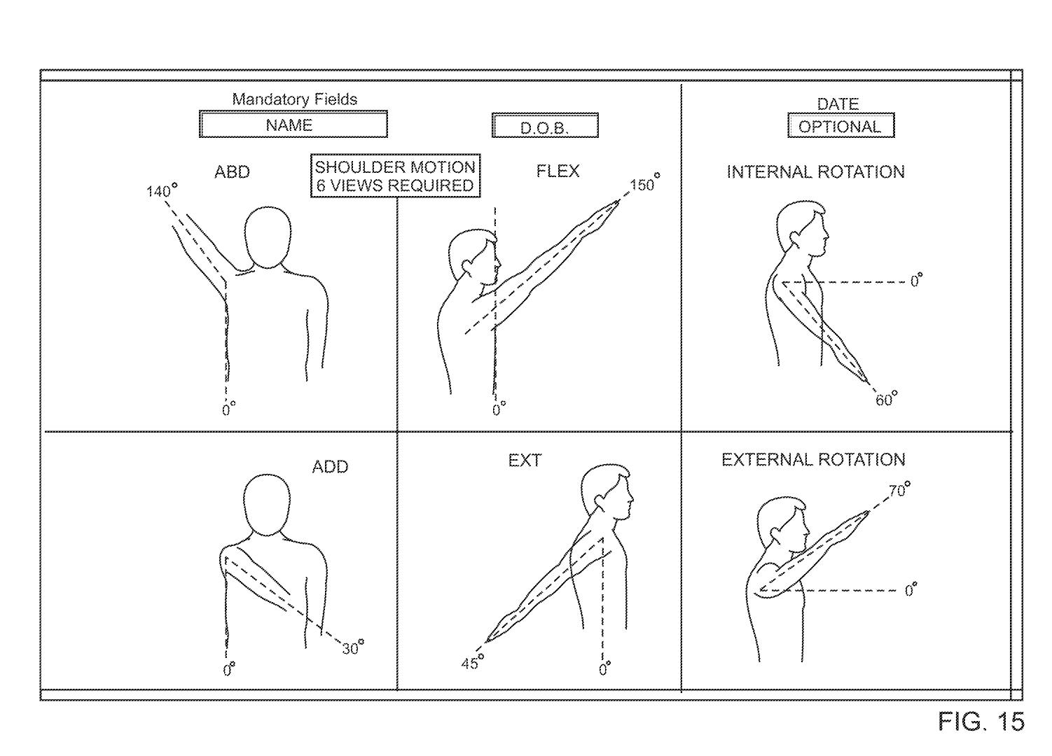

FIG. 15 illustrates a series of digital images provided on-screen for measuring and recording the degree of permanent impairment in shoulder motion. Six different views of the injured shoulder (left or right) are shown on-screen for measuring shoulder joint abduction, adduction, flexion, extension, internal rotation (IR) and external rotation (ER). Once again the ULIMS software provides an on-screen measurement tool, typically in the form of a protractor (not shown), for measuring the extent of the injury. As with the amputation assessment, the measurement tool can be moved over the digital image, and, the relevant angle of motion of the injured limb can be measured by locating the measurement tool on-screen so as overlay the injured shoulder.

This measurement can then be used to obtain the degree of impairment with reference to FIGS. 16-40, 16-43 and 16-46 in AMA 5^{th}, (or FIGS. 3.89, 3.41 and 3.44 in AMA 4^{th}), for calculating the degree of permanent shoulder motion impairments due to loss of abduction, loss of adduction, loss of extension, loss of flexion, loss of internal rotation, loss of external rotation, and ankylosis of the shoulder joint. Alternatively the ULIM software may store several look-up tables in the computer memory to automatically calculate the degree of permanent impairment. These values, together with the measured angles can be recorded in conjunction with the digital image and a hardcopy printed if desired as a permanent record.

A further advantage of having the ULIMS software calculate the degree of permanent impairment with reference to look-up tables, is that many of the Figures in AMA 5^{th} or AMA 4^{th} are in the form of pie charts with figures only provided for angular increments of 10°. Hence when using these pie charts it is necessary for the clinician to guesstimate or interpolate the values for angles falling between the angles shown on the pie chart. However the ULIMS takes the guesswork out of these calculations as it automatically performs a mathematical interpolation to calculate the exact value for the angle specified. The clinician may still round-up or round-down to the nearest whole number if required. The ULIMS also allows the vertical or horizontal reference axis to be accurately set as the zero value, which is difficult to do accurately by sight when using a handheld goniometer.

A feature of the ULIMS software is the provision of an on-screen measurement tool for measuring the extent of injury. A user may elect not to use the on-screen measurement tool and instead use an old fashioned manual protractor to measure the angles (of digit rotation, digit angulation, finger, thumb, wrist, elbow or shoulder motion) by measuring (and optionally drawing lines) on the photographs.

Now that a preferred embodiment of the method and apparatus for measuring upper limb permanent impairment has been described in detail, it will be apparent that the described embodiment provides a number of advantages over the prior art, including the following:
(i) It provides photographic evidence visible to all parties.
(ii) It provides a precise, verifiable, reproducible and transparent method that ensures accuracy or measurement and that is open to review.
(iii) All uncertainly and inaccuracy can be eliminated in such permanent impairment assessments, and a permanent record can be made available immediately as a ‘print out’ for clients, workers or 3^{rd} parties.
(iv) The same measurement apparatus and method can be used with a range of optional on-screen tools to measure related areas of digit amputation, digit or thumb rotation, digit angulation, sensory level, as well as wrist, elbow and shoulder motion impairment assessment which rely on a range of motion measurements.

It will be readily apparent to persons skilled in the relevant arts that various modifications and improvements may be made to the foregoing embodiments, in addition to those already described, without departing from the basic inventive concepts of the present invention. For example, whilst the described method of assessment makes reference to the AMA 5^{th} or 4^{th}, it will be apparent that the method of permanent impairment assessment can be readily adapted using other suitable reference standards to perform the actual calculations. Therefore, it will be appreciated that the scope of the invention is not limited to the specific embodiments described.

1. A method of permanent impairment assessment of a person's upper limb and/or hand, the method comprising the steps of:
displaying a digital image of the person's injured limb and/or hand and of their uninjured limb and/or hand on a visual display screen;
providing a measurement tool on-screen for measuring the extent of injury, wherein the measurement tool can be moved over the digital image;
measuring the extent of injury of the injured limb and/or hand by moving the measurement tool so as overlay the injured limb and/or hand; and, obtaining a value for the degree of impairment using this measurement.

2. A method of permanent impairment assessment as defined in claim 1, wherein the method further comprises the steps of:
identifying in the digital image key scaling markers on the uninjured limb and/or hand and any corresponding key scaling markers remaining on the injured limb and/or hand; and,
scaling the measurement tool using the key scaling markers on the uninjured limb and/or hand.

3. A method of permanent impairment assessment as defined in claim 2, wherein the method further comprises the step of the user physically marking the key scaling markers on both the uninjured hand and the injured hand, prior to the step of making the digital image to aid in the positive identification of such key scaling markers in the digital image.

4. A method of permanent impairment assessment as defined in claim 1, wherein the method further comprises the step of saving a digital record of the digital image with measurements superimposed on the image.

5. A method of permanent impairment assessment as defined in claim 1, wherein once the measurement tool has been scaled, the digital image of the uninjured limb and/or hand is marked with a visual scale indicator representative of the scale resolution.

6. A method of permanent impairment assessment as defined in claim 5, wherein the visual scale indicator is superimposed on the digital image of the uninjured limb and/or hand, and a permanent copy of this image is stored with the patient record to provide a visual indicator that the scale of the measurement tool remained unchanged between measurements.

7. A method of permanent impairment assessment as defined in claim 6, wherein an image of the measurement tool overlaying the injured limb and/or hand is also superimposed on the permanent copy of the digital image of the injured hand stored with the patient record.

8. A method of permanent impairment assessment as defined in claim 7, wherein the image of the measurement tool overlaying the injured limb and/or hand also includes the digital scale indicator, providing visual proof that the scale of the measurement tool was unchanged between measurements.

9. A method of permanent impairment assessment as defined in claim 1, wherein the method also comprises the step of enabling the user to select the types of measurement of permanent impairment it is desired to make and on which parts of the upper limbs and/or hands the measurements are to be made.

10. A method of permanent impairment assessment as defined in claim 9, wherein the method also comprises the step of indicating the number and types of views (digital images) required of the upper limbs and/or hands to perform the selected measurements.

11. An apparatus for permanent impairment assessment of a person's upper limb and/or hand, the apparatus comprising:
a visual display screen for displaying a digital image of the person's injured limb and/or hand and of their uninjured limb and/or hand;
digital processing means for processing the digital image and controlling the information displayed in the visual display screen;
an on-screen measurement tool for measuring the extent of the injury, wherein the measurement tool can be moved over the digital image; and,
wherein, in use, the extent of injury of the injured limb and/or hand can be measured by moving the measurement tool so as overlay the injured limb and/or hand and this measurement can be used to obtain a value for the degree of impairment.

12. An apparatus for permanent impairment assessment as defined in claim 11, wherein the apparatus further comprises means for identifying in the digital image key scaling markers on the uninjured limb and/or hand and any corresponding key scaling markers remaining on the injured limb and/or hand, and wherein the measurement tool can be scaled using the key scaling markers on the uninjured limb and/or hand.

13. An apparatus for permanent impairment assessment as defined in claim 11, wherein the apparatus further comprises imaging means for making a digital image of the person's injured limb and/or hand and of their uninjured limb and/or hand.

14. A method of permanent impairment assessment of a person's rotated digit or thumb, the method comprising the steps of:
having the person grip an elongate linear object with both hands;
measuring a first angle of rotation, if any, of the person's uninjured digit or thumb;
measuring a second angle of rotation of the person's injured digit or thumb; and,
subtracting the first angle from the second angle to obtain a measurement of the degree of rotation of the injured digit or thumb; and,
obtaining a value for the degree of impairment using this measurement.

15. A method of permanent impairment assessment of a person's rotated digit or thumb as defined in claim 14, the method further comprising the steps of:
displaying a digital image of the person's injured hand and of their uninjured hand gripping the elongate object on a visual display screen;
providing a measurement tool on-screen for measuring the angle of rotation, wherein the measurement tool can be moved over the digital image; and,
measuring the first and second angles of rotation of the uninjured digit or thumb and injured digit or thumb respectively by moving the measurement tool so as to first overlay the uninjured digit or thumb and then the injured digit or thumb.

16. A method of permanent impairment assessment of a person's rotated digit or thumb as defined in claim 15, wherein the measurement tool is in the form of an on-screen protractor which can be used to measure the angle of rotation of the injured thumb or digit shown in the image.

17. An apparatus for permanent impairment assessment of a person's rotated digit or thumb, the apparatus comprising:
an elongate linear object adapted to be gripped with both hands; and, means for measuring a first angle of rotation, if any, of the person's uninjured digit or thumb, and for measuring a second angle of rotation of the person's injured digit or thumb;
wherein, in use, by subtracting the first angle from the second angle to obtain a measurement of the degree of rotation of the injured digit or thumb, the degree of impairment can also be obtained using this measurement.

18. An apparatus for permanent impairment assessment of a person's rotated digit or thumb as defined in claim 17, wherein the elongate linear object is a cylindrical object that can be comfortably gripped with both hands.

19. An apparatus for permanent impairment assessment of a person's rotated digit or thumb as defined in claim 17, wherein the cylindrical object has an external diameter of between 40-50 mm.
